(19) United States
(12) Patent Application Publication
(10) Pub. No.: US 2025/0057485 A1
(43) Pub. Date: Feb. 20, 2025

(54) FALL RISK MITIGATION SYSTEM

(71) Applicant: Christiana Care Health System, Inc., Wilmington, DE (US)

(72) Inventors: Kristie Cudmore, Elkton, MD (US); Kerry D'Alessandro, Newark, DE (US); Jessica Dickerson, Chesapeake City, MD (US); Jonathan Meade, Newark, DE (US); Geoff Shertzer, Wilmington, DE (US)

(21) Appl. No.: 18/804,266

(22) Filed: Aug. 14, 2024

Related U.S. Application Data
(60) Provisional application No. 63/532,936, filed on Aug. 16, 2023.

(51) Int. Cl. A61B 5/00; A61B 5/11; A61G 7/05; G05B 19/042

(52) U.S. Cl. CPC A61B 5/7275; A61B 5/112; A61B 5/1126; A61B 5/746; A61G 7/0524; G05B 19/042; G05B 2219/2608

(57) ABSTRACT

A fall risk mitigation system includes a sensor gathering data associated with a person's bodily movements, and a control system that processes the sensor data and assesses the person's fall risk. The sensor may be integrated into a wearable device, or may be integrated into the person's environment as a camera/imaging device, a sensor in a floor tile or mat, etc., to gather movement data passively. If a heightened fall risk state is found, the control system may initiate a fall-mitigating intervention by transmitting a control signal to another device. The control signal may notify a caregiver, to prompt the caregiver to provide the intervention. Alternatively, the control signal may cause another device to notify the person, or cause a mechanism in the person's environment to automatedly deploy an assistive device to aid in preventing a fall, such as a bed guardrail, a handrail or grab bar.

### CROSS-REFERENCE TO RELATED APPLICATION

This application claims the benefit of priority under 35 U.S.C. § 119(e) of U.S. Provisional Patent Application No. 63/532,936, filed Aug. 16, 2023, the entire disclosure of which is hereby incorporated herein by reference.

### FIELD OF THE INVENTION

The present invention relates generally to prevention of falls that may result in injuries to patients and others, and more particularly, to a fall risk mitigation system that can passively observe and assess a patient as to fall risk, alert clinicians of a patient with a heightened fall risk, and adjust a level of environmental support based upon the current fall risk level.

### DISCUSSION OF RELATED ART

Elderly persons, patients recuperating post-surgery, and persons with various afflictions have a heightened risk of falling and sustaining injuries as a result of a fall. Hospitals, nursing homes, and other entities that care for patients are interested in reducing falls, and particularly in avoiding falls while a person is in their care.

There is a particularly acute risk of injury when a person has been identified as having a heightened fall risk or when the person's ability to ambulate safely in a given area degrades. In clinical settings, a conventional fall risk assessment is a clinician's manual, subjective evaluation of a user's fall risk as a result of clinical examination performed by the clinician. By its very nature, the clinical examination is performed at a particular point in time and is merely a snapshot of the person's fall risk at the time of this examination. Further, such a fall risk examination is performed only in the event that there are reasons to believe heightened fall risk factors are present, and accordingly, many persons are unexamined that may have a heightened fall risk, but do not show signs that lead to performance of a fall risk examination.

In the clinical context, after a person has been recognized to have a heightened fall risk, the heightened fall risk assessment stays in place until an additional assessment is performed to update the fall risk status. The manual nature of the assessment and updating process means that patients not displaying heightened risk factors but with a reduced ability to safely move in a given area could be missed. It is also possible that a degradation in the person's ability to ambulate safely may be missed after the initial assessment, or that fall precautions stay in place unnecessarily for a patient that has regained normalized ability and thus no longer has a heightened fall risk.

What is needed is a system that passively observes and objectively assesses a person's ambulation/walking ability to detect a movement pattern or degradation in movement/ambulation ability that indicates a heightened fall risk state, and that notifies clinicians and/or initiates fall-prevention safety precautions in response to a detection of such a heightened fall risk state.

### BRIEF DESCRIPTION OF THE FIGURES

An understanding of the following description will be facilitated by reference to the attached drawings, in which:

FIG. 1 is a perspective view of an exemplary fall risk mitigation system in accordance with an exemplary embodiment of the present invention shown in use in an exemplary environment;

FIG. 2 is a schematic diagram of a control system of the fall risk mitigation system of FIG. 1;

FIG. 3 is a flow diagram 500 illustrating an exemplary method for fall assessment ability evaluation in accordance with an exemplary embodiment of the present invention; and

FIG. 4 is a flow diagram illustrating an exemplary automated fall risk mitigation device activation workflow in accordance with the present invention.

### DETAILED DESCRIPTION

The present invention provides a monitoring system that performs an objective, ongoing assessment of a person's ability to safely ambulate/walk within an environment/space and changes the level of support and restriction a patient receives based on a passive assessment of each movement the person makes.

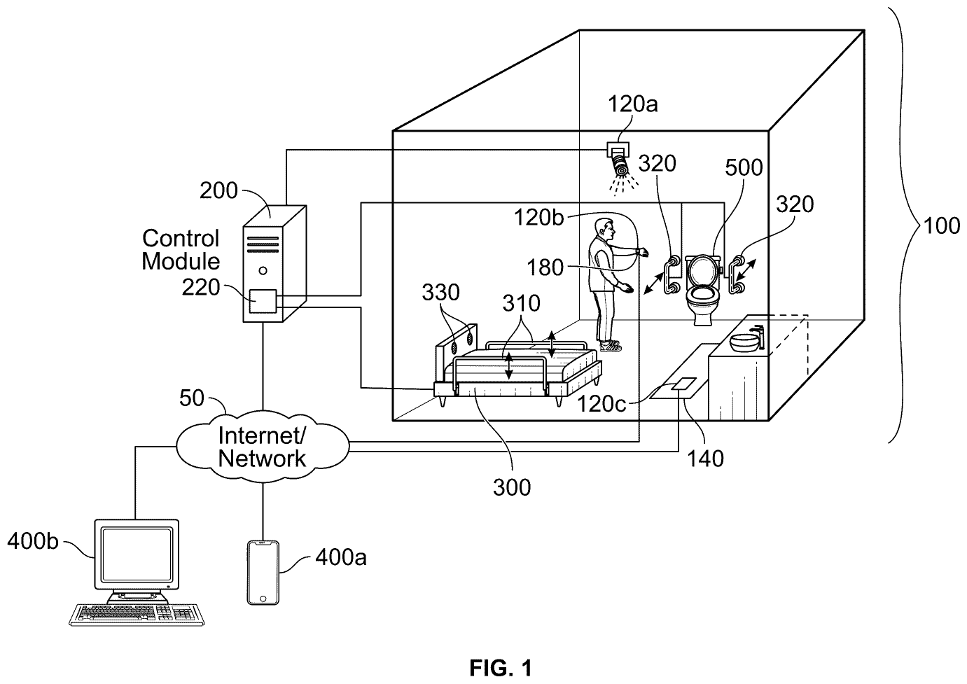

FIG. 1 is a perspective view of an exemplary fall risk mitigation system 100 in accordance with an exemplary embodiment of the present invention, shown in use in an exemplary environment 500. More particularly, the fall risk mitigation system 100 includes at least one sensor 120 for passively observing and objectively assessing bodily movements of a person P within a defined environmental space.

In one exemplary embodiment, the sensor 120 (e.g., 120a, 120b, 120c, etc.) may be a radar- or lidar-based sensor or a digital camera/imaging-based sensor 120a, which may be fixedly mounted (e.g., on a wall or ceiling) in the environment 500, as shown in the example of FIG. 1. For illustrative purposes only, this imaging sensor 120a is shown in the example of FIG. 1 as communicating directly with local/on-premise monitoring system 100 hardware, although alternatively, the sensor 120a or may communicate to a remote/cloud-based instance of the fall mitigation system via a communications network, such as the internet.

In other embodiments, one or more sensors (such as an accelerometer, gyroscope, etc.) 120b may be incorporated into a carryable (e.g., smartphone) or wearable device 180, as shown in the example of FIG. 1. By way of further example, in other embodiments, one or more sensors 120c may be incorporated into a flooring tile, floor mat or other floor covering 140 in the environment 500, as shown in the example of FIG. 1. For illustrative purposes only, these sensors 120b, 120c are shown in the example of FIG. 1 as communicating to a remote/cloud-based instance of the fall mitigation system 100 via a communications network, such as the internet, although alternatively, the sensors 120b, 120c may communicate directly with local/on-premise monitoring system 100 hardware.

The sensor 120 is positioned relative to the environment 500 (e.g., within the environment) so as to passively observe the person's movements, particularly, the person's ambulation/walking movements, within the environment. By way of example, the environment may be a person's room in the person's home, in a hospital, or in a nursing home, or in a common room/area of the person's home, of a hospital, or of a nursing home, or in another area.

By way of example, the fall mitigation system 100 uses passive observation via the sensor 120 (e.g., using machine vision or a less-intrusive radar/lidar monitoring technique) to gather data that is processed to determine whether a person is exhibiting movement that indicates a heightened fall risk.

The fall mitigation system 100 includes a control system 200. The control system 200 processes the data received from the sensor 120 to assess the person's ambulatory/walking ability, and to determine whether the observed ambulatory/walking behavior suggests that the person is presently in a state of heightened fall risk.

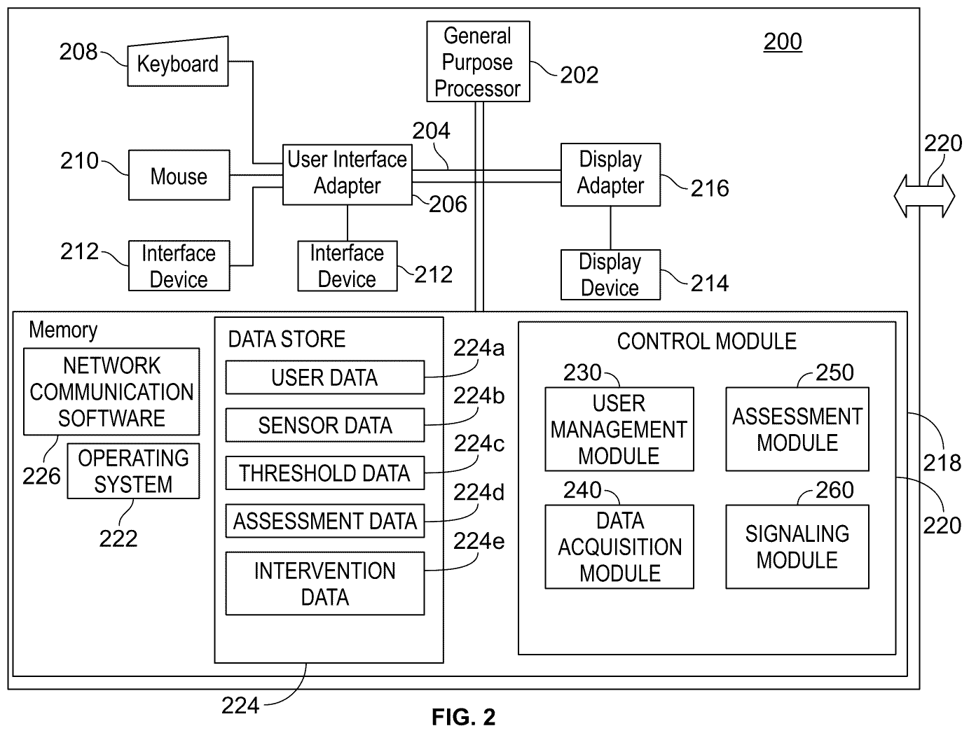

FIG. 2 is a schematic diagram of the control system (CS) 200 of the fall risk mitigation system of FIG. 1. The exemplary CS 200 includes conventional computing hardware storing and executing conventional software enabling operation of a general-purpose computing system, such as operating system software 222, network communications software 226. Additionally, the CS 200 stores and executes additional computer software for carrying out at least one method in accordance with the present invention. By way of example, the communications software 226 may include conventional web server software, and the operating system software 222 may include iOS, Android, Windows, Linux software.

Accordingly, the exemplary CS 200 of FIG. 2 includes a general-purpose processor, such as a microprocessor (CPU), 202 and a bus 204 employed to connect and enable communication between the processor 202 and the components of the control system in accordance with known techniques. The exemplary CS 200 includes a user interface adapter 206, which connects the processor 202 via the bus 204 to one or more interface devices, such as a keyboard 208, mouse 210, and/or other interface devices 212, which can be any user interface device, such as the sensor 120/120a/120b/120c, etc., a camera, microphone, touch sensitive screen, digitized entry pad, etc. In certain embodiments, the CS 200 may not include all of these interface devices. The bus 204 also connects a display device 214, such as an LCD screen or monitor, to the processor 202 via a display adapter 216. The bus 204 also connects the processor 202 to memory 218, which can include a hard drive, diskette drive, tape drive, etc.

The CS 200 may communicate with other devices, computers or networks of computers, for example via a communications channel, network card or modem 220. The CS 200 may be associated with such other computers in a local area network (LAN) or a wide area network (WAN), and may operate as a server in a client/server arrangement with another computer, etc. Such configurations, as well as the appropriate communications hardware and software, are known in the art.

The CS 200 includes computer-readable, processor-executable instructions stored in the memory 218 for carrying out the methods described herein. Further, the memory 218 stores certain data, e.g. in one or more databases or other data stores 224 shown logically in FIG. 2 for illustrative purposes, without regard to any particular embodiment in one or more hardware or software components.

Further, as will be noted from FIG. 2, the CS 300 includes, in accordance with the present invention, a Control Module (CM) 220, shown schematically as stored in the memory 218, which includes a number of modules providing functionality in accordance with the present invention, as discussed in greater detail below. These modules may be implemented primarily by software including microprocessor-executable instructions stored in the memory 218 of the CS 200. Optionally, other software may be stored in the memory 218 and and/or other data may be stored in the data store 224 and/or memory 218.

As referenced above, the control system 200 processes the data received from the sensor(s) 120 to assess the person's ambulatory/walking ability, e.g., to determine whether the observed ambulatory/walking behavior suggests that the person is presently in a state of heightened fall risk and in certain cases to initiate a mitigating intervention, either indirectly by prompting a caregiver to provide the intervention, or directly by transmitting a control signal control to a fall risk mitigation device, e.g., to activate/deploy the fall risk mitigation device, as described in greater detail below.

Accordingly, as shown in FIG. 2, the exemplary control module 220 of the exemplary control system 200 includes a User Management Module (UMM) 230. The UMM 230 is responsible for displaying graphical user interface windows for gathering person/patient identification data, associated sensor/device data, etc., to effectively create, store and/or retrieve a personal profile within the system, e.g., to permit the control system 200 to provide its functionality on a personalized basis for each of many different persons. Accordingly, the UMM 220 may retrieve data from and/or store data in the Data Store 224 as User Data 224a.

Further, as shown in FIG. 2, the exemplary control module 220 of includes a Data Acquisition Module (DAM) 240. The DAM 240 is responsible for receiving sensor data from the sensor(s) 120/120a/120b/120c, etc. (e.g., as may be communicated directly from a local device/sensor and/or via a data communications network 50, such as the Internet), and storing such data in the Data Store 224 as Sensor Data 224b. The Sensor Data 224b may be stored in a manner to associate it with a particular person to which it pertains, as that person is identified in the User Data 224a. This allows the received Sensor Data 224b for a particular person to be processed to perform a fall risk assessment of that person using the applicable Sensor Data 224b.

Further still, as shown in FIG. 2, the exemplary control module 220 of includes an Assessment Module (AM) 250. The AM 250 is responsible for processing sensor data from the sensor(s) 120/120a/120b/120c, etc. (which may involve use of the general-purpose processor 202 and/or retrieving of appropriate sensor Data 224b from the Data Store 224). More particularly, AM 250 is responsible for processing the sensor data to perform an assessment of the person's fall risk and/or determining whether the person is in a heightened fall risk state.

By way of example, the AM 250 may be configured to update a computerized risk model with captured data from each patient movement observed by the sensor 120 in the environment 500 to allow for detection and alerting of a degradation of walking/standing ability indicating a heightened fall risk state.

The AM's determination of whether the person is presently in a heightened fall risk state may involve determination of whether the sensor 120 has gathered data consistent with fall risk indicators. Fall risk indicators may include the identification of uneven, unsteady, unbalanced movement from one side of the body to the other or one leg to the other, as well as tripping, dragging of feet, shuffling, and any other gait or bodily movement that may indicate fall risk. The AM 250 may use these identified risk events to establish a risk model for the user, prioritizing the latest evaluation, repeated actions, and event risk potential (for instance, tripping producing a higher risk potential compared to a shuffle) to trigger alert or support actions. Any appropriate risk model, or other suitable structured manner of performing a risk assessment may be used, as will be appreciated by those skilled in the art.

Accordingly, the AM 250 may receive observation data from the sensor 120 in relation to the person, and processes the sensor data in accordance with predetermined instructions stored in a memory of the control module 220 to determine whether the person is in a heightened risk state.

In certain embodiments, an overall assessment of the person's ambulatory/walking ability/steadiness is reflected in a single metric, e.g., as a score, on a scale ranging from very unstable to very stable, and there is at least one predetermined threshold score representing a point at which intervention is needed. In such an embodiment, the AM 250 receives observation data from the sensor 120 in relation to the person, and processes the sensor data in accordance with its predetermined instructions to calculate the score. In this exemplary embodiment, the AM 250 may store the calculated score (or any other result of the assessment) as Assessment Data 224d in the Data Store 224.

By way of example, there may be different threshold scores associated with different needs for different types of interventions, as may be reflected in Threshold Data 224c stored in the Data Store 224. In such an embodiment, the AM 250 may compare a current score to a threshold score retrieved from the Threshold Data 224c stored in the memory, as a standard/benchmark to determine whether the person is currently in the heightened risk state, such that an intervention is presently warranted.

By way of further example, in certain embodiments, the person's current calculated score may be compared by the AM 250 to a prior score for the person (that was calculated and stored as prior Assessment Data 224d) to indicate improvement or degradation of walking ability/steadiness, and such comparison may be used to determine when to initiate an intervention. In certain embodiments, different scores may be deemed to warrant different interventions as a function of the calculated score.

Accordingly, the person is continuously/repeatedly monitored and assessed by the AM 250/control module 220/sensor 120/system 100 over time, as the person navigates the environment 500, etc. The recurring assessments allow for consideration of changes in the person's ambulatory ability over time, (e.g., over a long time relative to the assessment interval, such as a period of weeks, months, years or more), to allow for detection of minor changes in improvement or degradation of the person's ambulatory/walking ability and associated fall risk.

In this example, the control module 220 is further configured to allow safety measures to be put in place proactively as a precautionary measure until a clinician can examine the person, at which point the clinician may confirm or cancel the precautionary safety measures as a result of the clinician's examination. More particularly, the control module 220 is further configured to initiate an intervention so a fall can be prevented, or the risk of fall can be mitigated, in the event of a determination by the control module 220 that the observed ambulatory/walking behavior of the person indicates that the person is presently in a heightened fall risk state.

Accordingly, in this example shown in FIG. 2, the control module 220 further includes a Signaling Module (SM) 260. The SM 260 is responsible for initiating a mitigating intervention, and may transmit a signal using Intervention Data 224e stored in the Data Store 224 in association with the particular person.

In certain embodiments, the SM 260 is configured to initiate an intervention by sending a control signal to another device (which may be within or outside of the person's environment) that changes states to provide the intervention, in accordance with Intervention Data 224e stored in the Data Store 224. For example, the SM 260 may send the control signal to the person's bed/bed accessory 300 to drive a motor to cause it to actuate a fall risk mitigation device, such as to raise motorized bed guardrails 310, to a motor to extend motorized handrails/grab bars 320, or to activate bed alarms 330 that sound when the person leaves the bed or is preparing to leave the bed. By way of example, the SM 260 may send the control signal via wired or wireless data transmission directly to an electronic/computerized device, or via a data communications network 50, such as the internet and/or a cellular/mobile data network.

In certain embodiments, the SM 260 is configured to initiate an intervention by sending a control signal to another device 400 (such as a smartphone, laptop/tablet/PC computer, etc.) that provides an alert to a person who can intervene, in accordance with Intervention Data 224e stored in the Data Store 224 (such as contact/network information for sending the alert to the person). For example, the SM 260 may send a control signal to cause display of a message or other indicator, or sounding of an alarm, at a nursing station, or a caregiver's computer, smartphone or other computing device, to alert the recipient of the heightened fall risk so the person can take appropriate action, such as manually setting bedding guard rails, manually setting movement alarms, etc. For example, the message may be displayed on a display device of the other device 400, e.g., as a banner notification, email, or text/SMS/MMS message, or otherwise.

Appropriate sensors may be incorporated into clothing or wearable devices, or otherwise not be coupled to a specific room/environment. For example, in a home or mobile version of this system, computerized risk models may receive and use as inputs any sensor's data that can capture data indicative of a heightened fall risk state. For example, wearable devices or sensors in clothing could be used by the fall mitigation system to establish and communicate risk to a user's support person(s).

In certain embodiments, the SM 260 is configured to directly and automatedly activate proactive fall-precautions for a user until a clinician can confirm or negate necessity of the fall precaution, in accordance with Intervention Data 224e stored in the Data Store 224. Accordingly, instead of providing a control signal that provides a notification to a person such that the person can implement an appropriate intervention, in certain embodiments, the SM 260 may transmit the control signal to a device to control operation of the device such that the device itself provides an appropriate intervention, e.g., without the need for human involvement. For example, devices in hospital rooms, nursing home rooms, and/or for at-home consumer devices could be configured to react to the control signal in the event of the control module's fall risk assessment to change its functionality based on the fall mitigation system's determination of a heightened (or lessened) fall risk state. For instance, motorized/movable handrails/grab bars 320 of a bed, in a stairway, in a bathroom, etc. could be caused by the control signal to extend for the person's use to safely navigate an area. More particularly, if a heightened fall risk is identified by the fall mitigation system, the SM 260 then issues/transmits control signals to manipulate objects or aspects of the environment to assist the person in avoiding a fall—including existing consumer home automation products. This may include turning on lights in a particular area, issuing a voice-based reminder to hold a handrail or other assistive device, or automatically extending handrails/grab bars and/or seats for the person, e.g., in addition to notifying a caregiver or other support person.

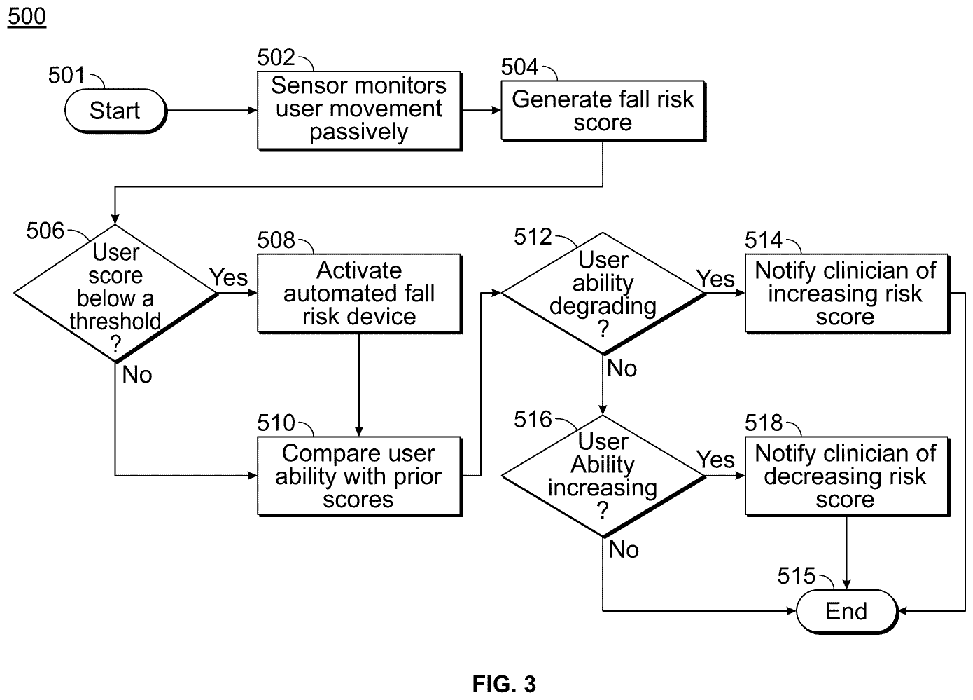

FIG. 3 is a flow diagram 500 illustrating an exemplary method for fall assessment ability evaluation in accordance with an exemplary embodiment of the present invention. As shown in FIG. 3, the exemplary method starts with the sensor 120 passively monitoring the user during movement/ambulation, as shown at 502 and 504. This results in the DAM 240 gathering and storing sensor data, as described above.

Next, the exemplary method involves generating a fall risk score reflecting observed ambulation ability and gait evaluation, as shown at 506. This may be performed by the AM 250 using received/stored Sensor Data 224c, as described above.

Next, the exemplary method involves determining whether the user's fall risk score is below a safe level, as shown at 506. This may be performed by the AM 250 using Threshold Data 224c stored in the Data Store 224, as described above.

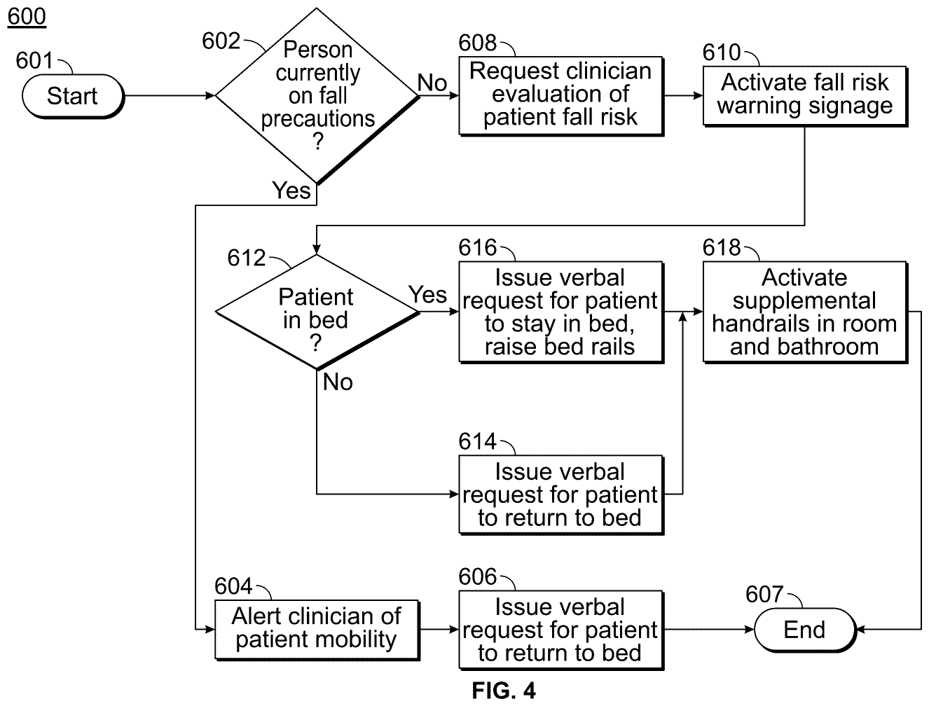

In this exemplary embodiment, if the fall risk score is determined by the AM 250 to be below a threshold at 506, then a Fall Risk Device intervention is activated, as shown at 508, Accordingly, in this exemplary embodiment, an intervention is initiated in this case by the AM 250, as described in greater detail in with reference to FIG. 4.

In this exemplary embodiment, if the fall risk score is determined by the AM 250 not to be below the threshold at 506, then the AM 250 compares the user's current fall risk score with a prior calculated fall risk score, as shown at 510. If the AM 250 determines that the person's ability is degrading at 512 (because the current fall risk score is worse/higher that the person's prior score), then the AM 250 causes the Signaling Module 260 to transmit a control signal to notify the clinician (e.g., using clinician contact information for the person stored in the Intervention Data 224e) of the increasing fall risk score/worsening condition, as shown at 514, and the method ends, as shown at 515.

In this exemplary embodiment, if the AM 250 determines at 512 that the person's ability is not degrading at 512 (because the current fall risk score is worse/higher that the person's prior score), then the AM 250 determines whether the person's ability is increasing (because the current fall risk score is better/lower than the person's prior score), as shown at 516.

If the person's ability is determined by the AM 250 not to be increasing, then the method ends, as shown at 516 and 515. If, however, the person's ability is determined by the AM 250 to be increasing at 515, then the AM 250 causes the Signaling Module 260 to transmit a control signal to notify the clinician (e.g., using clinician contact information for the person stored in the Intervention Data 224e) of the decreasing fall risk score/improving condition, as shown at 518, and the method ends, as shown at 515.

As shown in the exemplary embodiment of FIG. 3, the fall risk mitigation system 100 is used to determine whether there is a fall risk. If it is determined that the person's fall risk score is at or above a fall risk threshold (as described with reference to FIG. 3.), then method flow continues to FIG. 4. FIG. 4 is a flow diagram 500 illustrating an exemplary method for automated fall risk device activation in accordance with an exemplary embodiment of the present invention. As shown in FIG. 4, the exemplary method starts with the AM 250 determining whether the person is currently on fall precautions, as shown at 601 and 602. This may involve the AM 250 reference data stored as User Data 224a and/or Assessment Data 224d.

If the person is already on fall precautions, then the exemplary method involves the AM 250 causing the SM 260 to transmit a control signal to alert the person's clinician (e.g., using retrieved Intervention Data 224e for the person) of the person's mobility (when the sensor indicates mobility as determined by the AM 250), as shown at 604. In this case, the clinician may deliver a mitigating intervention, e.g. by issuing a verbal request to the patient, asking the patient to return to bed, and the method ends, as shown at 606 and 607.

If, however, the AM 250 determines that the person is not currently on fall precautions, as shown at 602, then the exemplary method involves the AM 250 causing the SM 260 to transmit a control signal to alert the person's clinician (e.g., using retrieved Intervention Data 224e for the person) requesting the clinician to perform an evaluation of the patient's fall risk, as shown at 608.

Further, this exemplary method involves the AM 250 causing the SM 260 to transmit a control signal to an external device (e.g., using retrieved Intervention Data 224e for the person) activating/illuminating fall risk warning signage in the person's room, etc., as shown at 610.

This exemplary method involves the AM 250 next determining whether the sensor data indicates that the person is presently in bed, as shown at 612.

If not, then the AM 250 causes the SM 260 to transmit a control signal to an external device (e.g., using retrieved Intervention Data 224e for the person) issuing an audible instruction to return to bed in the person's room, etc., as shown at 614.

If so, then the AM 250 causes the SM 260 to transmit a control signal to an external device (e.g., using retrieved Intervention Data 224e for the person) issuing an audible instruction to stay in bed in the person's room, etc., as shown at 616, and further causes the SM 260 to transmit a control signal to an external device to activate a fall risk mitigation device, such as a bed's guardrails, supplemental handrails/grab bars in a bedroom or bathroom, etc., as shown at 618, and the method ends, as shown at 607.

Accordingly, the control system 200 processes the data received from the sensor(s) 120 to assess the person's ambulatory/walking ability, e.g., to determine whether the observed ambulatory/walking behavior suggests that the person is presently in a state of heightened fall risk and can initiate a mitigating intervention, either indirectly by prompting a caregiver to provide the intervention, or directly by transmitting a control signal control to a fall risk mitigation device, e.g., to automatedly activate/deploy the fall risk mitigation device.

While there have been described herein the principles of the invention, it is to be understood by those skilled in the art that this description is made only by way of example and not as a limitation to the scope of the invention. Accordingly, it is intended by the appended claims, to cover all modifications of the invention which fall within the true spirit and scope of the invention.

What is claimed is:

1. A fall risk mitigation system comprising:
a sensor configured to passively observe bodily movements of a person and gather data associated with such bodily movements; and
a control system operable to process the data gathered by said sensor and to assess the person's fall risk as a function of the gathered sensor data.

2. The fall risk mitigation system of claim 1, wherein said sensor is selected from a group consisting of a camera-based sensor, a radar-based sensor, and a lidar-based sensor.

3. The fall risk mitigation system of claim 1, wherein said sensor is selected from a group consisting of an accelerometer and a gyroscope.

4. The fall risk mitigation system of claim 1, wherein said sensor is integrated into a wearable device.

5. The fall risk mitigation system of claim 1, wherein said sensor is integrated into at least one of a flooring tile, a floor mat, a floor covering and a flooring product.

6. The fall risk mitigation system of claim 1, wherein said control system is configured to compare current movement data to prior movement data to detect a degradation of ambulation ability of the person.

7. The fall risk mitigation system of claim 1, wherein said control system is configured to detect at least one of unbalanced movement of the body, tripping, dragging of feet, and shuffling of feet.

8. The fall risk mitigation system of claim 1, wherein said control system is configured to express said assessment of the person's fall risk in a single metric as a fall risk score on a scale ranging from a very unstable score to a very stable score.

9. The fall risk mitigation system of claim 8, wherein said control system is configured to compare the person's fall risk score to a threshold score.

10. The fall risk mitigation system of claim 1, wherein said control system is configured to process the sensor data to determine whether the person is currently in a heightened fall risk state.

11. The fall risk mitigation system of claim 1, wherein said control system is configured to process the sensor data to determine whether the person is currently in a heightened fall risk state, and to initiate a mitigating intervention by transmitting a control signal to another device.

12. The fall risk mitigation system of claim 11, wherein said control system is configured to initiate the mitigating intervention by transmitting a control signal to another device to raise guardrails of a bed.

13. The fall risk mitigation system of claim 11, wherein said control system is configured to initiate the mitigating intervention by transmitting a control signal to another device to extend a handrail for grasping by the person.

14. The fall risk mitigation system of claim 11, wherein said control system is configured to initiate the intervention by transmitting a control signal to another device to cause at least one of a lighting device and sign to illuminate, and a voice-based reminder and audible alarm to be audibly produced.

15. The fall risk mitigation system of claim 11, wherein said control system is configured to initiate the intervention by transmitting a signal to a computing device of a caregiver to cause display of an indicating prompting the caregiver to provide the mitigation intervention.

16. The fall risk mitigation system of claim 1, wherein said control system comprises:
a processor,
a memory operative connected to said processor;
data communication hardware operable to receive sensor data and transmit control signals; and
a control system module comprising instructions stored in said memory and executable by said processor to process the sensor data to:
assess the person's fall risk; and
cause the data communication hardware to transmit a control signal as a function of the assessed fall risk.

17. A fall risk mitigation system comprising:
a sensor configured to passively observe bodily movements of a person and gather data associated with such bodily movements; and
a control system operable to process the data gathered by said sensor and to assess the person's fall risk as a function of the gathered sensor data, said control system comprising:
a processor;
a memory operative connected to said processor;
data communication hardware operable to receive sensor data and transmit control signals; and
a control system module comprising instructions stored in said memory and executable by said processor to process the sensor data to:
assess the person's fall risk; and
cause the data communication hardware to transmit a control signal as a function of the assessed fall risk.

18. The fall risk mitigation system of claim 17, wherein said sensor is selected from a group consisting of a camera-based sensor, a radar-based sensor, a lidar-based sensor, an accelerometer, and a gyroscope.

19. The fall risk mitigation system of claim 17, wherein said control system is configured to transmit the control signal to another device to drive a motor to perform at least one or raising guardrails of a bed and extending at least one of a handrail and a grab bar.

20. The fall risk mitigation system of claim 17, wherein said control system is configured to transmit the control signal to another device to cause at least one of a lighting device and sign to illuminate, and a voice-based reminder and audible alarm to be audibly produced.

21. The fall risk mitigation system of claim 17, wherein said control system is configured to transmit the control signal to another device to cause display of an indicating prompting the caregiver to provide the mitigation intervention.

22. A fall risk mitigation system comprising:
a motor operable to selective extend and retract at least one of a guardrail of a bed, a handrail and a grab bar;
a sensor configured to passively observe bodily movements of a person and gather data associated with such bodily movements; and
a control system operable to process the data gathered by said sensor and to assess the person's fall risk as a function of the gathered sensor data, said control system comprising:
a processor;
a memory operatively connected to said processor;
data communication hardware operable to receive sensor data and transmit control signals; and
a control system module comprising instructions stored in said memory and executable by said processor to process the sensor data to:
assess the person's fall risk to determine whether the person is in a heightened fall risk state; and
cause the data communication hardware to transmit a control signal to drive said motor to extend said at least one of said guardrail of the bed, the handrail and the grab bar if the person is assessed to be in the heightened fall risk state, and to drive said motor to retract said at least one of said guardrail of the bed, the handrail and the grab bar if the person is assessed to not be in the heightened fall risk state.
